(19) United States
(12) Patent Application Publication
(10) Pub. No.: US 2011/0213263 A1
(43) Pub. Date: Sep. 1, 2011

(54) METHOD FOR DETERMINING A HEARTBEAT RATE

(75) Inventor: Jacobus Cornelis Haartsen, Hardenberg (NL)

(73) Assignee: SONY ERICSSON MOBILE COMMUNICATIONS AB, Lund (SE)

(21) Appl. No.: 12/713,781

(22) Filed: Feb. 26, 2010

(51) Int. Cl. A61B 5/02

(52) U.S. Cl. 600/528

(57) ABSTRACT

A method for determining a heartbeat rate of a user from an acoustic heartbeat signal, a heartbeat rate measuring device, and a mobile device are described.

### BACKGROUND OF THE INVENTION

The present invention relates to a method for determining a heartbeat rate of a user from an acoustic heartbeat signal detected in an ear of the user and a heartbeat rate measuring device for detecting a heartbeat rate of a user from an acoustic heartbeat signal detected in the ear of the user.

### BRIEF SUMMARY OF THE INVENTION

According to an embodiment, a method for detecting a heartbeat rate of a user is provided. The heartbeat rate is determined from an acoustic heartbeat signal. The acoustic heartbeat signal is detected in an ear of the user. According to the method, the heartbeat signal is sampled with a predefined sampling rate. Furthermore, a plurality of heartbeat rate values are provided. According to the method, the sampled heartbeat rate signal is correlated with each of the plurality of heartbeat rate values. Then, the heartbeat rate value with the best matching correlation to the sampled heartbeat signal is determined and this heartbeat rate value is assigned as the heartbeat rate of the user.

When a user of a mobile device, for example a mobile telephone, a personal digital assistant, a mobile navigation system, a mobile media player or a mobile computer, is doing exercises or workout, for example jogging, walking, climbing, or biking, the user may listen to music being played back by the mobile device during exercise. Furthermore, the user may be interested in his or her current heartbeat rate to control the training. A mobile device supporting the above-described method of the present invention is adapted to determine the heartbeat rate by detecting the acoustic heartbeat signal in the ear of the user. This can be simply accomplished by a microphone integrated into an in-ear headphone providing the music for the user. According to the above-described embodiment of the present invention, a correlation technique is used to determine the current heartbeat rate of the user and thus the technique is very robust against for example noise or disturbances. Furthermore, the technique can be carried out at a very low sampling rate which results in a low power consumption and a low amount of processing power of the mobile device.

According to an embodiment, the plurality of heartbeat rate values is based on the predefined sampling rate. By choosing the heartbeat rate values based on the predefined sampling rate, a high correlation between the sampled heartbeat rate signal and the thus provided heartbeat rate values can be achieved which makes the technique even more robust.

Furthermore, the detected heartbeat signal may be filtered with a low pass filter. The low pass filter may have a cut off frequency in a range from 4 Hz to 10 Hz, preferably 5 Hz. Furthermore, an absolute value of the detected heartbeat signal may be formed. Filtering and forming an absolute value may remove noise and disturbing signals to make the heartbeat rate determination more robust.

According to another embodiment, correlating the sampled heartbeat signal with each of the plurality of heartbeat rate values comprises for each heartbeat rate value the following: For each heartbeat rate value a plurality of cumulation values are provided. The number of the plurality of cumulation values is defined by the heartbeat rate value and the predefined sampling rate. When a new sample value of the sampled heartbeat signal is sampled this sample value is added to a cumulation value of the plurality of cumulation values. Thereby, consecutive sample values of the sampled heartbeat signal are added to consecutively and cyclicly addressed cumulation values.

Thus, for each heartbeat rate value a list of cumulation values is provided which is addressed consecutively and cyclicly. A new sample value is added to the currently addressed cumulation value of each of the heartbeat rate values. Then, the next cumulation value is addressed for each of the heartbeat rate values. The length of each list, i.e. the number of cumulation values for each list, is different for each heartbeat rate value and defined by the heartbeat rate value and the predefined sampling rate. When a detected heartbeat rate signal correlates with a heartbeat rate value, in the corresponding cumulation value list some of the cumulation values will grow very strong whereas other cumulation values of the same list will grow very slow. When the detected heartbeat signal does not correlate to the heartbeat rate value, in the corresponding list of cumulation values the cumulation values will grow more uniformly. Thus, the cumulation values clearly indicate a correlation between the heartbeat signal and the corresponding heartbeat rate value. Performing the above-described correlation requires only very little computing power and can therefore be performed on a mobile device having restrictions due to processing power and energy.

According to an embodiment, determining the heartbeat rate value with the best matching correlation comprises a determining of a difference value between a minimum cumulation value and a maximum cumulation value of the plurality of cumulation values relating to the heartbeat rate value for each of the heartbeat rate values. Thus, for each of the heartbeat rate values a separate difference value is determined. Then, the maximum difference value of these difference values is determined and the heartbeat rate value with the maximum difference value is defined as the heartbeat rate value which correlates best to the detected heartbeat signal of the user.

According to an embodiment, the number of the plurality of cumulation values for each heartbeat rate value is defined by a ratio of the predefined sampling rate to the heartbeat rate value.

According to another embodiment, a cumulation value is reduced by a predetermined factor before adding a sample value to the cumulation value. The value of the predetermined factor may be in the range of 0.8 to 0.95, preferably 0.9. By reducing the cumulation value before adding a new sample value a “forget” function is realized which allows that the correlation automatically adapts continuously to a varying heartbeat rate of the user.

The sampling rate for sampling the acoustic heartbeat signal may be in the range of 10 to 200 Hz, preferably 22.05 Hz.

According to another embodiment, a heartbeat rate measuring device is provided. The device comprises a microphone adapted to detect an acoustic heartbeat signal of a user in an ear of the user, a sampling unit adapted to sample the heartbeat signal with a predefined sampling rate, and a correlation unit. The correlation unit provides a plurality of heartbeat rate values and is adapted to correlate the sampled heartbeat signal with each of the plurality of heartbeat rate values and to determine the heartbeat rate value with the best matching correlation.

According to an embodiment, the plurality of heartbeat rate values is based on the predefined sampling rate. The predetermined sampling rate may be in the range of 10 to 200 Hz, preferably 22.05 Hz.

The heartbeat measuring device may comprise furthermore a low pass filter adapted to filter the detected heartbeat signal. The low pass filter may have a cut off frequency in a range from 4 Hz to 10 Hz, preferably 5 Hz.

Furthermore, the heartbeat rate measuring device may comprise an absolute value unit adapted to form an absolute value of the detected heartbeat signal.

According to an embodiment, the correlation unit is adapted to provide for each heartbeat rate value a plurality of cumulation values. The number of the plurality of cumulation values is defined by the heartbeat rate value and the predefined sampling rate. The correlation unit may be further adapted to add a sample value of the sampled heartbeat signal to a cumulation value of the plurality of cumulation values for each heartbeat rate value. Consecutive sampling values are added to consecutively and cyclicly addressed cumulation values for each heartbeat rate value.

Furthermore, the correlation unit may be adapted to determine for each heartbeat rate value a difference value defining a difference between a minimum cumulation value and a maximum cumulation value of the plurality of cumulation values relating to the heartbeat rate value, and to define the heartbeat rate value with a maximum difference value as the heartbeat rate value with the best matching correlation.

The number of the plurality of cumulation values for each heartbeat rate value may be defined by a ratio of the predefined sampling rate to the heartbeat rate value.

According to an embodiment, the correlation unit is adapted to reduce the cumulation value by a predetermined factor before adding the sample value to the cumulation value. The predetermined factor may be in the range from 0.8 to 0.95, preferably 0.9.

According to an embodiment, a mobile device is provided. The mobile device comprises a heartbeat rate measuring device. The heartbeat rate measuring device comprises a microphone adapted to detect an acoustic heartbeat signal of a user in an ear of the user, a sampling unit adapted to sample the heartbeat signal with a predefined sampling rate, and a correlation unit providing a plurality of heartbeat rate values. The correlation unit is adapted to correlate the sampled heartbeat signal with each of the plurality of heartbeat rate values, and to determine the heartbeat rate value with the best matching correlation.

The mobile device may comprise a mobile phone, personal digital assistant, a mobile navigation system, a mobile media player, or a mobile computer.

Although specific features described in the above summary and the following detailed description are described in connection with specific embodiments, it is to be understood that the features of the embodiments described can be combined with each other unless it is noted otherwise.

### BRIEF DESCRIPTION OF THE SEVERAL VIEWS OF THE DRAWINGS

Hereinafter, exemplary embodiments of the invention will be described with reference to the drawings.

FIG. 1 shows the relation between human heart activities and heart sounds and a signal of an electrocardiogram.

FIG. 2 is a schematic diagram of a heartbeat rate measuring device according to an embodiment of the present invention.

FIG. 3 is a schematic diagram showing details of the heartbeat rate device of FIG. 2.

FIG. 4 shows a heartbeat signal detected in an ear of a user.

FIG. 5 shows the absolute value of the heartbeat signal of FIG. 4.

FIG. 6 shows a filtered heartbeat signal of the heartbeat signal of FIG. 5.

FIG. 7 shows a principle of correlating the sampled heartbeat signal against different heartbeat rate values having impulse streams which differ in period and in time offset.

FIG. 8 shows a correlation matrix which may be used to implement the correlation according to the present invention.

FIG. 9 shows an output of a heartbeat rate measuring device according to the present invention in response to a changing heartbeat rate of a user.

FIGS. 10-14 each show a correlation between a varying heartbeat rate of a user and a specific heartbeat rate value.

FIG. 15 shows cumulation values for different heartbeat rate values.

### DETAILED DESCRIPTION OF THE INVENTION

In the following, exemplary embodiments of the present invention will be described in detail. It is to be understood that the following description is given only for the purpose of illustrating the principles of the invention and is not to be taken in a limiting sense. Rather, the scope of the invention is defined only by the appended claims and not intended to be limited by the exemplary embodiments hereinafter.

It is to be understood that the features of the various exemplary embodiments described herein may be combined with each other unless specifically noted otherwise.

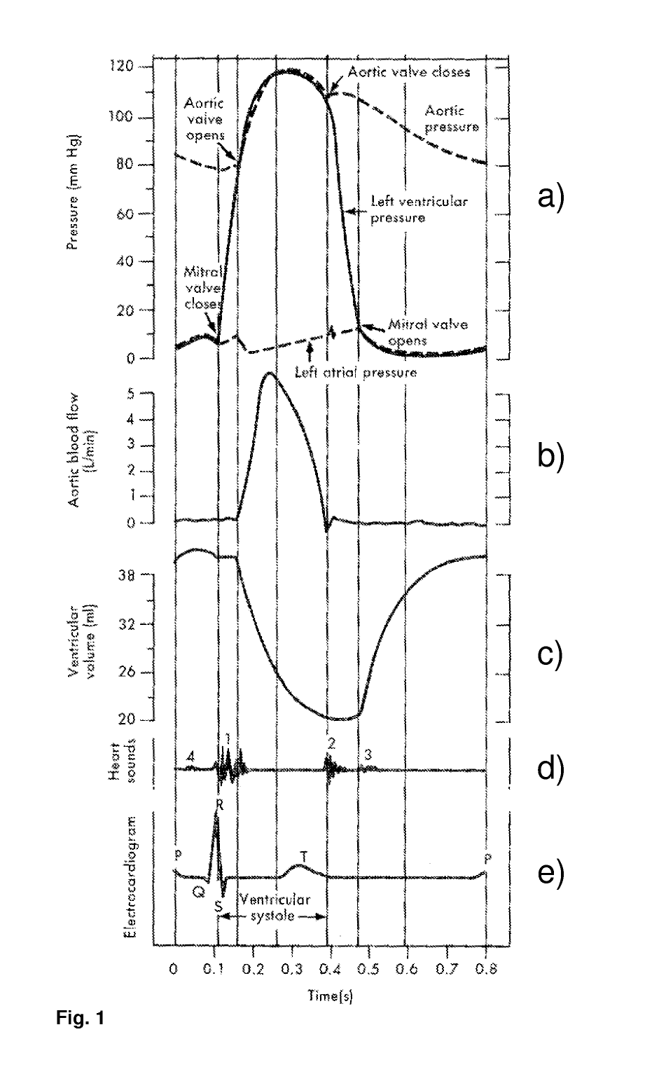

FIG. 1 shows activities occurring during a heartbeat of a human heart and the resulting heart sounds and resulting signals detected by an electrocardiogram. In FIG. 1a) a blood pressure during a heartbeat is shown. FIG. 1b) and FIG. 1c) show the corresponding aortic blood flow and ventricular volume, respectively, during the heartbeat. FIG. 1d) shows the resulting heart sounds which may be detected for example with a phonocardiogram (PCG). The corresponding electric heart signal which may be detected by an electrocardiogram (ECG) is shown in FIG. 1e). The electrocardiogram signal clearly shows the known P-wave QRS-complex T-wave ECG signal. The dual acoustic pulse observed in FIG. 1d) is caused by the opening and closing of the aortic walls. An audio signal detected with an in-ear microphone may be used to detect an acoustic pulse signal similar to the one shown in FIG. 1d) in the ear of a human being. This allows for example a user of a mobile device wearing a headphone with an in-ear microphone to observe his or her heartbeat rate. However, the audio signal detected in the ear of the user may be disturbed by surrounding noise and sound from the headphone loudspeakers. Therefore, the present invention provides a robust heartbeat rate detection from an audio signal detected in the ear of the user.

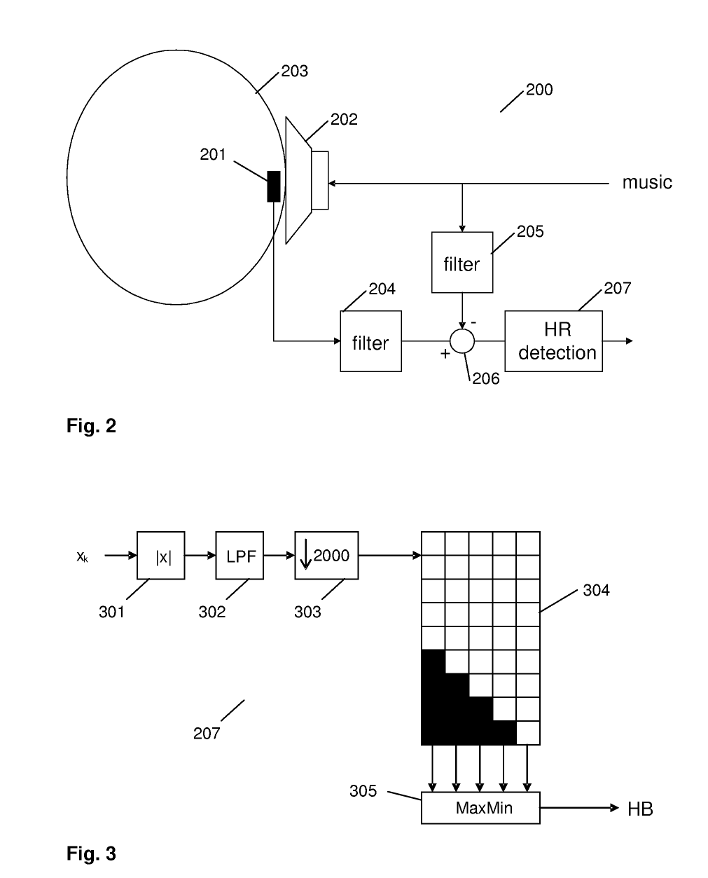

FIG. 2 shows a schematic view of an embodiment of a heartbeat rate measuring device. The heartbeat rate measuring device 200 comprises a microphone 201 which may be located together with a headphone loudspeaker 202 in an ear 203 of a user. The headphone loudspeaker 202 is used for playing back music or communication data to the user. The microphone 201 receives in the ear of the user the acoustic heartbeat signal together with music from the headphone loudspeaker 202. The signal from the microphone 201 is forwarded to a first filter 204 to render only the low frequency part where the heartbeat signal resides. From this frequency part the music signal provided to the headphone loudspeaker 202 is subtracted via a second filter 205 and a subtracting unit 206. The resulting signal is forwarded to a heartbeat rate detection unit (HR detection) 207.

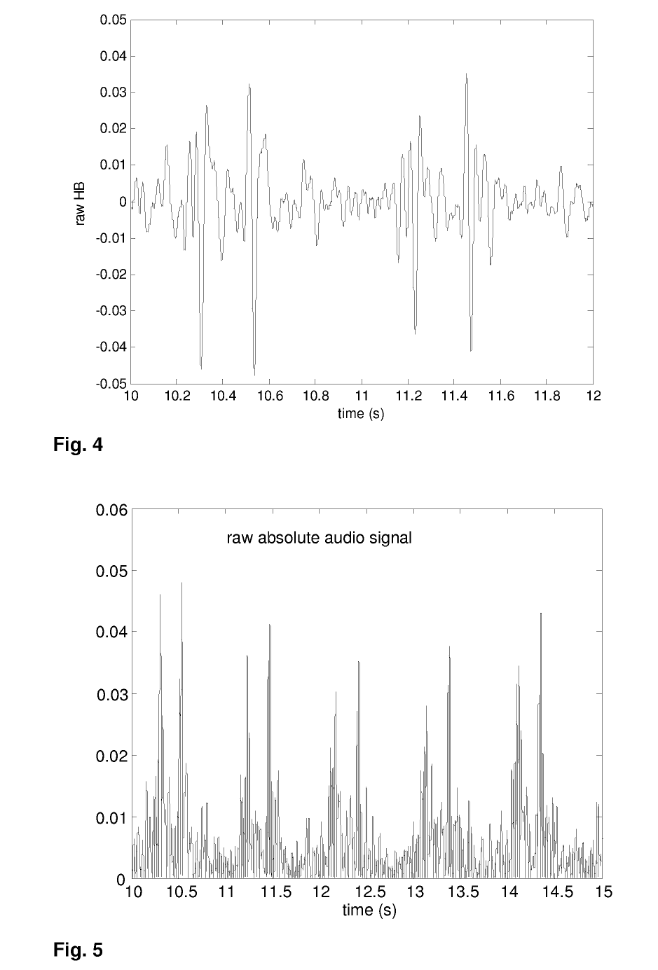

As described in connection with FIG. 1, the acoustic signal derived from the heartbeat is only loosely coupled to the electric signal (ECG) from the heart. The acoustic signal detected in the ear results from the blood flow in the arteries. Instead of the easy detectable electrocardiogram signal (ECG) shown in FIG. 1e) a sampled and filtered signal detected in an ear of the user as described above may look like the graph shown in FIG. 4. The dual bursts which correspond to the dual acoustic pulse shown in FIG. 1d) can be clearly observed. In contrast to the electrocardiogram signal which has a clean, recurrent waveform, the waveform of the acoustic signal is not stable. It is hard to find a template or reference signal for the acoustic waveform.

Therefore, the heartbeat rate detection unit 207 performs a detection based on an energy detection of the audio waveform. This allows the signal to be greatly downsampled as the pulse beat resides only at very low frequencies, for example 5 Hz and below. This reduces the amount of signal processing and thus offers low power implementations.

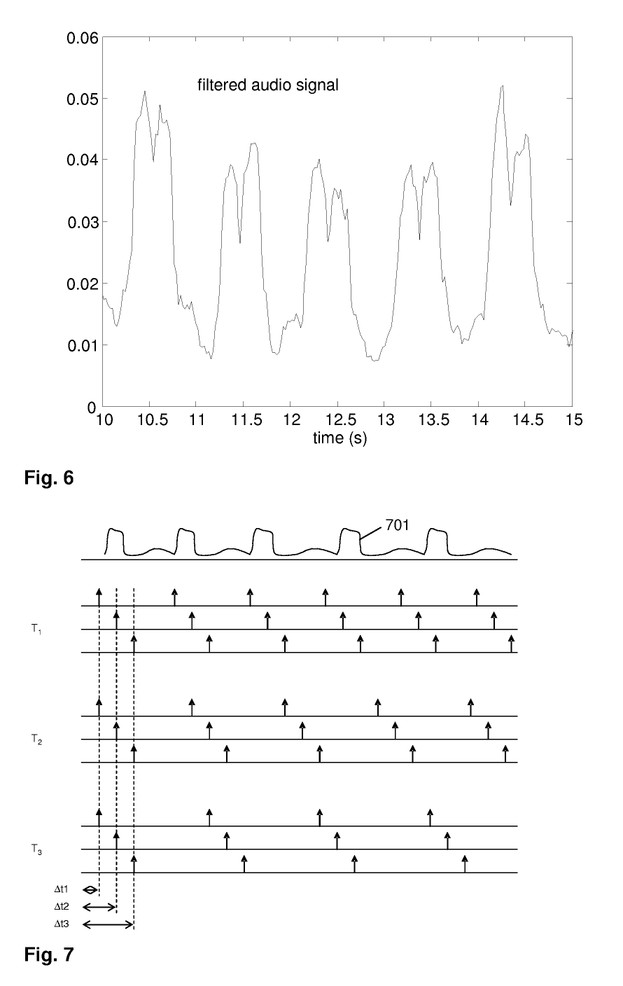

FIG. 3 shows a block diagram of an embodiment of the heartbeat rate detection unit 207. Since the detection is based on energy, first the absolute value of the sampled signal xₖ is taken in an absolute value unit 301. Then, the sampled audio signal is filtered with a low pass filter 302 (LPF) and downsampled with a downsampling unit 303. The low pass filter 302 may be a moving average filter with a first zero at 5 Hz. The signal may be downsampled to a rate of 22.05 Hz. FIG. 4 shows the signal xₖ before the absolute value unit 301, FIG. 5 shows the signal between the absolute value unit 301 and the low pass filter 302, and FIG. 6 shows the output of the low pass filter 302. Due to the low pass filtering the signal may be downsampled by a factor of 2000 with the down-sampling unit 303, from for example 44.1 kHz to 22.02 Hz.

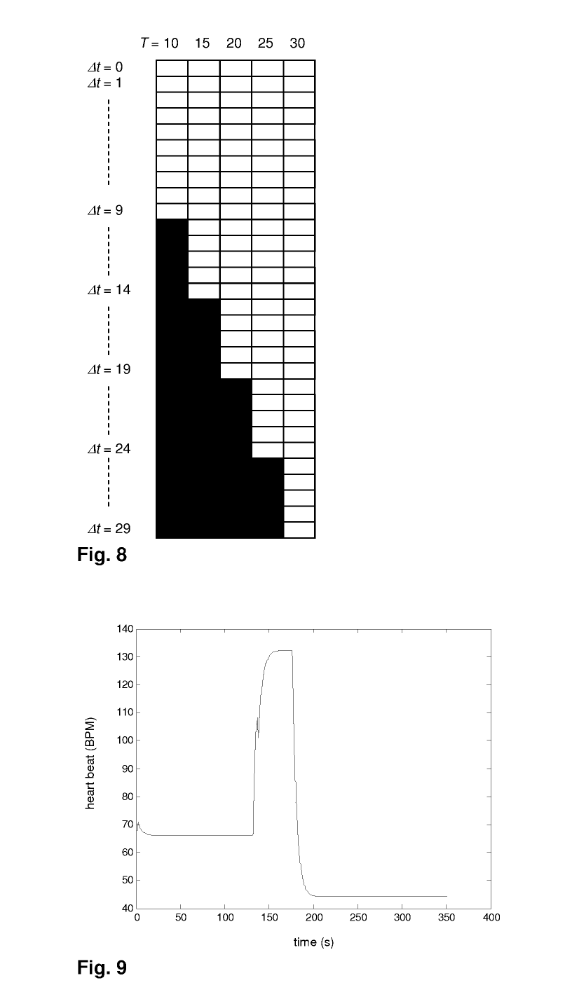

For a robust detection the downsampled audio signal is correlated against a series of impulse streams which differ in period (interval) and in time offset. FIG. 7 shows this principle. The heartbeat rate signal 701 is correlated against impulse streams with different periods (T₁, T₂, T₃) and different time offsets (Δt1, Δt2, Δt3). This correlation can be done on the fly using a matrix in which the columns represent the different periods and the rows represent the different time offsets. This function is performed in the heartbeat detection unit 207 of FIG. 3 by a correlation unit 304 which will be described in more detail in connection with FIG. 8. The correlation unit 304 comprises a cumulation value matrix as shown in FIG. 8. In the matrix shown in FIG. 8 the first row represents a time offset of zero, the second row represents a time offset of one sample (for example at a 22.05 Hz sampling rate this corresponds to an offset of 45.3 ms), the third row represents the time offset of two samples (90.6 ms) and so on. The first column represents a period T₁ of 10 samples (which means ten times 45.3 ms=453 ms which would correspond to a heartbeat rate of 132 beats per minute), the second column a period T₂ of 15 samples (corresponding to 88 beats per minute), the third column a period T₃ of 20 samples (corresponding to 66 beats per minute), the fourth column a period T₄ of 25 samples (corresponding to 53 beats per minute) and the fifth column a period T₅ of 30 samples (corresponding to 44 beats per minute). This is just an illustrative example and in other embodiments more rows and columns may be taken in order to increase the resolution of the heartbeat rate measurement.

Whenever a new downsampled value of the heartbeat signal is provided by the downsampling unit 303 to the correlation unit 304 (FIG. 3) one element in each column of FIG. 8 is updated according to the following equation:

W(mod(k,Tᵢ),i)=αW(mod(k,Tᵢ),i)+(1−α)xₖ

wherein

W(r, c) is the matrix element of row r with r in the range from 0 to Tᵢ−1 and column c with a range of 1 to 5 in the example of FIG. 8,

mod(a, b) is the modulo operation a mod b,

k is a counter counting the input samples starting at 0,

Tᵢ is the period of the considered column with i in the range of 1 to 5 in the example of FIGS. 8 and T₁=10, T₂=15, T₃=20, T₄=25 and T₅=30,

α is a predetermined factor, and

xₖ is the value of sample k of the downsampled heartbeat signal.

Thus, each column of the matrix of FIG. 8 contains a different number of values corresponding to the period of the considered column. Column 1 contains 10 values, column 2 contains 15 values, column 3 contains 20 values and so on. In FIG. 8 the not used values in the matrix are indicated by being filled with black color. With the modulo operation the rows of each column are addressed consecutively and cyclicly. For example, when a new value x₀ is sampled, in each column the value of the first row is modified according to the above-described function. With the next sample value x₁ the values of the second row are modified. This continues until the 11^{th} sample value x₁₀ arrives. The value x₁₀ is then used for modifying the first row of column 1 and the 11^{th} row of columns 2-5. Thus, the sampled audio signal is correlated against the different heartbeat rate values assigned to the columns of the matrix of FIG. 8.

The modification or update process defined in the above-described equation uses an exponential forget function with the parameter α to average between new and past samples. For a large α more averaging will be carried out and correlation over more energy bursts will be applied. However, the system will have a slower response to a change in the heartbeat rate. α may be in the range of 0.8 to 0.95, preferably 0.9.

When modifying the matrix values, one column of FIG. 8 that has a period matching best to the current heartbeat of the user will become stable with high maximum values for that time offset for which the impulse streams aligns with the energy bursts, and low minimum values for that time offset for which the impulse stream falls halfway between the energy bursts. Therefore, for each column of the matrix of FIG. 8 a difference value Wₘₐₓ_{_{—}}ₘᵢₙ defining a difference between the maximum value W_{MAX} of the column and the minimum value W_{MIN} of the column is determined by the MaxMin-unit 305 of FIG. 3. Furthermore, the MaxMin-unit 305 determines for which of the columns this difference value Wₘₐₓ_{_{—}}ₘᵢₙ is the largest and defines the heartbeat rate value of the column with the largest difference value as the heartbeat rate of the user.

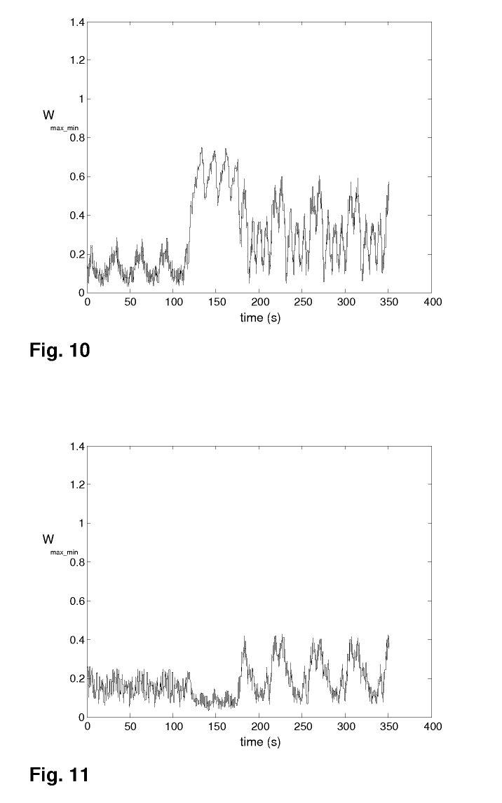

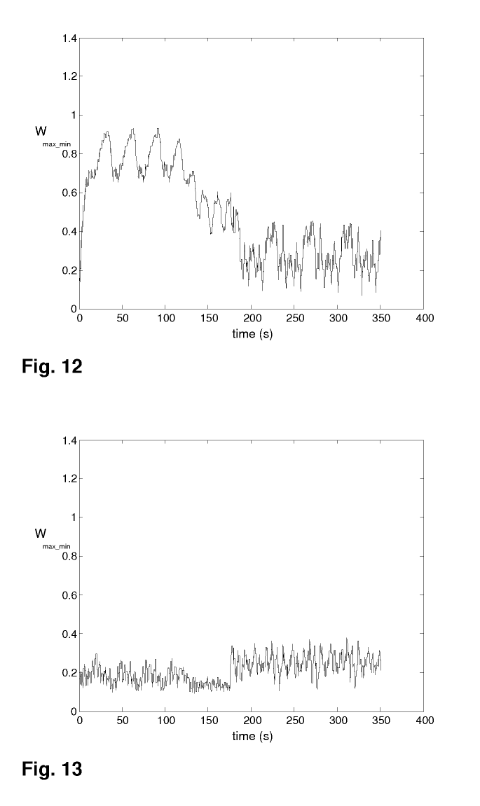

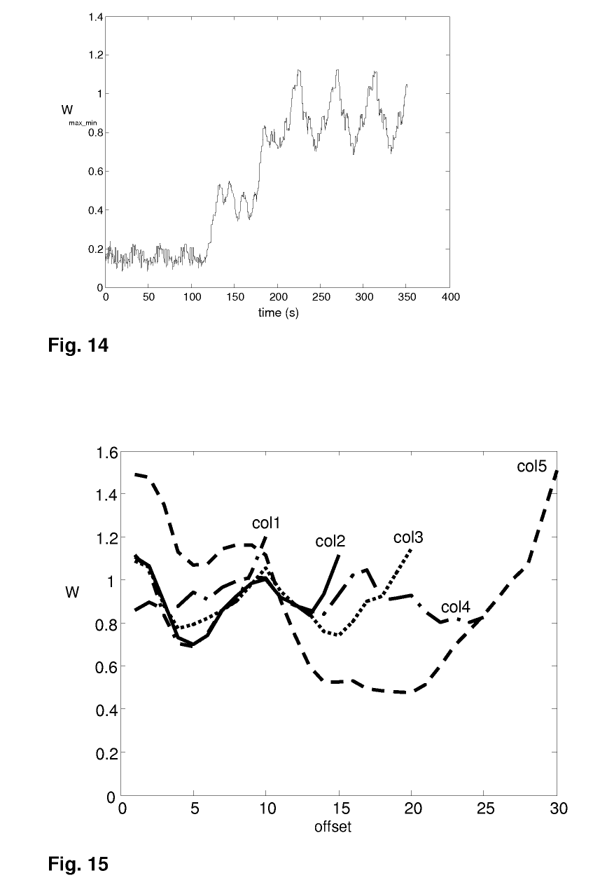

Operation of the heartbeat rate measuring device as shown in connection with FIGS. 2 and 3 and explained as described above, will be now described with the help of an example. A test signal is created where the heartbeat rate is varied between 66 beats per minute in a first section, 132 beats per minute in a second section and 44 beats per minute in a third section. FIG. 9 shows the response of the above-described heartbeat rate detection unit 207 for α=0.9. On the x-axis of FIG. 9 the time is shown in seconds and on the y-axis of FIG. 9 the determined heartbeat rate is shown. FIGS. 10-14 show the developing of the value Wₘₐₓ_{_{—}}ₘᵢₙ=W_{MAX}−W_{MIN} for each of the columns of FIG. 8. FIG. 10 shows the developing of Wₘₐₓ_{_{—}}ₘᵢₙ for the first column, FIG. 11 shows the developing of Wₘₐₓ_{_{—}}ₘᵢₙ for the second column, FIG. 12 shows the developing of Wₘₐₓ_{_{—}}ₘᵢₙ for the third column, FIG. 13 shows the developing of Wₘₐₓ_{_{—}}ₘᵢₙ for the fourth column, and FIG. 14 shows the developing of Wₘₐₓ_{_{—}}ₘᵢₙ for the fifth column of FIG. 8. By comparing the FIGS. 10-14 it can be clearly seen that one column always provides a larger Wₘₐₓ_{_{—}}ₘᵢₙ than the others. For the first section of the test signal from second 0 to approximately second 120 column 3 (FIG. 12) representing a heartbeat rate of 66 beats per minute provides the largest Wₘₐₓ_{_{—}}ₘᵢₙ compared to the other columns. In the second section from approximately second 120 to approximately second 160 the first column (FIG. 10) provides the largest Wₘₐₓ_{_{—}}ₘᵢₙ corresponding to a heartbeat rate of 132 beats per minute. In the last section of the test signal starting approximately at second 160 the last column (FIG. 14) provides the largest values Wₘₐₓ_{_{—}}ₘᵢₙ indicating a heartbeat rate of 44 beats per minute.

Finally, FIG. 15 shows a snapshot of the five columns col1, col2, col3, col4, col5 in the matrix shown in FIG. 8. Each graph for each column shows the accumulated values in the columns for the different offsets. Column col5 provides the largest difference between W_{MAX} and W_{MIN}. Since column col5 corresponds to T_{S}=30 which corresponds to a period of 1.36 seconds, this corresponds to a heartbeat rate of approximately 44 beats per minute.

The above-described algorithm is very robust and operates even when noise and disturbances are added to the acoustic heartbeat signal. The heartbeat rate is based on a number of consecutive energy bursts and gaps between the energy bursts. For further noise suppression, filtering is applied by an exponential “forget” function characterized by an “forget” parameter α. For larger α the method will be more robust at the expense of a longer latency and a slower response speed.

As in the above-described method very robust correlation techniques are used, the described method is very robust against disturbances and noise. Furthermore, the described techniques are carried out at a very low sampling rate which results in a low power consumption. Furthermore, the amount of processing power is very low. Therefore, the above-described method and the above-described heartbeat rate measuring device can be advantageously integrated into a mobile device, for example a mobile phone or a mobile reproduction device.

While exemplary embodiments have been described above, various modifications may be implemented in other embodiments. For example, the sampling rate after the downsampling unit 303 may be adapted to meet the requirements of a desired range and resolution for the heartbeat rate detection. Furthermore, the number of columns and rows in the matrix of FIG. 8 may be adapted accordingly.

Finally, it is to be understood that all the embodiments described above are considered to be comprised by the present invention as it is defined by the appended claims.

What is claimed is:

1. A method for determining a heartbeat rate of a user from an acoustic heartbeat signal, the method comprising:
detecting the acoustic heartbeat signal in an ear of the user,
sampling the heartbeat signal with a predefined sampling rate,
providing a plurality of heartbeat rate values,
correlating the sampled heartbeat signal with each of the plurality of heartbeat rate values, and
determining the heartbeat rate value with the best matching correlation as the heartbeat rate.

2. The method according to claim 1, wherein providing the plurality of heartbeat rate values is based on the predefined sampling rate.

3. The method according to claim 1, comprising low pass filtering of the detected heartbeat signal.

4. The method according to claim 3, wherein the low pass filtering comprises a cut-off frequency in a range from 4 Hz to 10 Hz.

5. The method according to claim 1, comprising forming an absolute value of the detected heartbeat signal.

6. The method according to claim 1, wherein correlating the sampled heartbeat signal with each of the plurality of heartbeat rate values comprises for each heartbeat rate value:
providing a plurality of cumulation values, wherein the number of the plurality of cumulation values is defined by the heartbeat rate value and the predefined sampling rate, and
adding a sample value of the sampled heartbeat signal to a cumulation value of the plurality of cumulation values, wherein consecutive sampling values are added to consecutively and cyclicly addressed cumulation values.

7. The method according to claim 6, wherein determining the heartbeat rate value with the best matching correlation comprises:
determining for each heartbeat rate value a difference value between a minimum cumulation value and a maximum cumulation value of the plurality of cumulation values relating to the heartbeat rate value, and
defining the heartbeat rate value with a maximum difference value as the heartbeat rate value with the best matching correlation.

8. The method according to claim 6, wherein the number of the plurality of cumulation values for each heartbeat rate value is defined by a ratio of the predefined sampling rate to the heartbeat rate value.

9. The method according to claim 6, wherein before adding the sample value to the cumulation value the cumulation value is reduced by a predetermined factor.

10. The method according to claim 9, wherein the value of the predetermined factor is in the range from 0.8 to 0.95.

11. The method according to claim 1, wherein the value of the predefined sampling rate is in the range from 10 to 200 Hz.

12. A heartbeat rate measuring device, comprising:
a microphone adapted to detect an acoustic heartbeat signal of a user in an ear of the user,
a sampling unit adapted to sample the heartbeat signal with a predefined sampling rate, and
a correlation unit providing a plurality of heartbeat rate values, the correlation unit being adapted to correlate the sampled heartbeat signal with each of the plurality of heartbeat rate values, and to determine the heartbeat rate value with the best matching correlation.

13. The heartbeat rate measuring device according to claim 12, wherein the plurality of heartbeat rate values is based on the predefined sampling rate.

14. The heartbeat rate measuring device according to claim 12, comprising a low pass filter adapted to filter the detected heartbeat signal.

15. The heartbeat rate measuring device according to claim 14, wherein the low pass filter has a cut-off frequency in a range from 4 Hz to 10 Hz.

16. The heartbeat rate measuring device according to claim 12, comprising an absolute value unit adapted to form an absolute value of the detected heartbeat signal.

17. The heartbeat rate measuring device according to claim 12, wherein the correlation unit is adapted to:
provide for each heartbeat rate value a plurality of cumulation values, wherein the number of the plurality of cumulation values is defined by the heartbeat rate value and the predefined sampling rate, and
add a sample value of the sampled heartbeat signal to a cumulation value of the plurality of cumulation values for each heartbeat rate value, wherein consecutive sampling values are added to consecutively and cyclicly addressed cumulation values.

18. The heartbeat rate measuring device according to claim 17, wherein the correlation unit is adapted to
determine for each heartbeat rate value a difference value between a minimum cumulation value and a maximum cumulation value of the plurality of cumulation values relating to the heartbeat rate value, and
define the heartbeat rate value with a maximum difference value as the heartbeat rate value with the best matching correlation.

19. The heartbeat rate measuring device according to claim 17, wherein the number of the plurality of cumulation values for each heartbeat rate value is defined by a ratio of the predefined sampling rate to the heartbeat rate value.

20. The heartbeat rate measuring device according to claim 17, wherein the correlation unit is adapted to reduce the cumulation value by a predetermined factor before adding the sample value to the cumulation value.

21. The heartbeat rate measuring device according to claim 20, wherein the value of the predetermined factor is in the range from 0.8 to 0.95.

22. The heartbeat rate measuring device according to claim 12, wherein the value of the predefined sampling rate is in the range from 10 to 200 Hz.

23. A mobile device comprising a heartbeat rate measuring device, the heartbeat rate measuring device, comprising:
a microphone adapted to detect an acoustic heartbeat signal of a user in an ear of the user,
a sampling unit adapted to sample the heartbeat signal with a predefined sampling rate, and
a correlation unit providing a plurality of heartbeat rate values, the correlation unit being adapted to correlate the sampled heartbeat signal with each of the plurality of heartbeat rate values, and to determine the heartbeat rate value with the best matching correlation.

24. The mobile device according to claim 23, wherein the mobile device comprises a device selected from the group comprising a mobile phone, a personal digital assistant, a mobile navigation system, a mobile media player, and a mobile computer.
